(19) United States
(12) Patent Application Publication
(10) Pub. No.: US 2022/0210586 A1
(43) Pub. Date: Jun. 30, 2022

(54) AUTOMATIC TINNITUS MASKER FOR AN EAR-WEARABLE ELECTRONIC DEVICE

(71) Applicant: Starkey Laboratories, Inc., Eden Prairie, MN (US)

(72) Inventors: Paul Reinhart, Minneapolis, MN (US); Tim Schoof, Minneapolis, MN (US)

(21) Appl. No.: 17/544,749

(22) Filed: Dec. 7, 2021

Related U.S. Application Data
(60) Provisional application No. 63/131,950, filed on Dec. 30, 2020.

(51) Int. Cl. H04R 25/00; G01P 13/00; G01J 1/42

(52) U.S. Cl. CPC H04R 25/75; H04R 25/65; H04R 25/604; H04R 2225/41; H04R 25/505; G01P 13/00; G01J 1/4204; H04R 25/609

(57) ABSTRACT

An ear-wearable electronic device comprises a housing configured to be worn in, at or about an ear of a wearer. A sound generator is disposed in the housing and configured to produce at least a tinnitus masking sound. A physiologic sensor arrangement is disposed in or supported by the housing and configured to measure one or both of a plurality of physiologic parameters and a plurality of physiologic conditions of the wearer. The physiologic sensor arrangement is configured to produce physiologic sensor signals in response to the measurements. A controller is operatively coupled to the sound generator and the physiologic sensor arrangement. The controller is configured to detect one or more of presence, absence, and severity of tinnitus of the wearer using the physiologic sensor signals.

### RELATED APPLICATIONS

This application claims the benefit of U.S. Provisional Application No. 63/131,950, filed Dec. 30, 2020, the content of which is hereby incorporated by reference.

### TECHNICAL FIELD

This application relates generally to ear-level electronic devices and systems, including hearing devices, personal amplification devices, hearing aids, bone conduction hearing devices, medical and consumer hearables, in-ear electronic appliances, electronic ear plugs, physiologic monitoring devices, biometric devices, position and/or motion sensing devices, and other ear-wearable electronic devices.

### SUMMARY

Some embodiments are directed to an ear-wearable electronic device comprising a housing configured to be worn in, at or about an ear of a wearer, and a sound generator disposed in the housing and configured to produce at least a tinnitus masking sound. A physiologic sensor arrangement is disposed in or supported by the housing and configured to measure a plurality of physiologic parameters or physiologic conditions of the wearer, the physiologic sensor arrangement configured to produce physiologic sensor signals in response to the physiologic sensor measurements. A controller is operatively coupled to the sound generator and the physiologic sensor arrangement, the controller configured to detect one or more of presence, absence, and severity of tinnitus of the wearer using the physiologic sensor signals.

Some embodiments are directed to an ear-wearable electronic device comprising a housing configured to be worn in, at or about an ear of a wearer, and a sound generator disposed in the housing and configured to produce at least a tinnitus masking sound. A physiologic sensor arrangement is disposed in or supported by the housing and configured to measure at least one physiologic parameter or at least one physiologic condition of the wearer, the physiologic sensor arrangement configured to produce physiologic sensor signals in response to the physiologic sensor measurement. A non-physiologic sensor arrangement comprises one or more non-physiologic sensors configured to sense a least one non-physiologic parameter or condition impacting a current context or wellbeing of the wearer, the non-physiologic sensor arrangement configured to produce non-physiologic sensor signals in response to the non-physiologic sensor measurement. A controller is operatively coupled to the sound generator, the physiologic sensor arrangement, and the non-physiologic sensor arrangement, the controller configured to detect one or more of presence, absence, and severity of tinnitus of the wearer using the physiologic sensor signals and the non-physiologic sensor signals.

Some embodiments are directed to an ear-wearable electronic device comprising a housing configured to be worn in, at or about an ear of a wearer, and a sound generator disposed in the housing and configured to produce at least a tinnitus masking sound. A communication device is disposed in or supported by the housing and configured to wirelessly communicate with an external electronic device or system and to receive, from the external electronic device or system, contextual factor data indicative of one or more factors impacting a current context or wellbeing of the wearer. A physiologic sensor arrangement is disposed in or supported by the housing and configured to measure at least one physiologic parameter or at least one physiologic condition of the wearer, the physiologic sensor arrangement configured to produce physiologic sensor signals in response to the measurement. A non-physiologic sensor arrangement comprises one or more non-physiologic sensors configured to sense at least one non-physiologic parameter or condition impacting a current context or wellbeing of the wearer, the non-physiologic sensor arrangement configured to produce non-physiologic sensor signals in response to the non-physiologic sensor measurement. A controller is operatively coupled to the sound generator, the communication device, the physiologic sensor arrangement, and the non-physiologic sensor arrangement, the controller configured to detect one or more of presence, absence, and severity of tinnitus of the wearer using the physiologic sensor signals, the non-physiologic sensor signals, and the contextual factor data.

Some embodiments are directed to a method implemented by an ear-wearable electronic device worn by a wearer and comprising measuring, using a physiologic sensor arrangement of the device, a plurality of one or both of physiologic parameters and physiologic conditions of the wearer, producing, by the physiologic sensor arrangement, physiologic sensor signals in response to the physiologic sensor measurements, and detecting, using a controller of the device, one or more of presence, absence and severity of tinnitus of the wearer using the physiologic sensor signals.

Some embodiments are directed to a method implemented by an ear-wearable electronic device worn by a wearer and comprising measuring, using a physiologic sensor arrangement of the device, at least one physiologic parameter or at least one physiologic condition of the wearer, producing, by the physiologic sensor arrangement, physiologic sensor signals in response to the physiologic sensor measurement, measuring, using a non-physiologic sensor arrangement of the device, at least one non-physiologic parameter or at least one condition impacting a current context or wellbeing of the wearer, producing, by the non-physiologic sensor arrangement, non-physiologic sensor signals in response to the non-physiologic sensor measurement, and detecting, using a controller of the device, one or more of presence, absence, and severity of tinnitus of the wearer using the physiologic sensor signals and the non-physiologic sensor signals.

Some embodiments are directed to a method implemented by an ear-wearable electronic device worn by a wearer comprising measuring, using a physiologic sensor arrangement of the device, at least one physiologic parameter and or at least one physiologic condition of the wearer, producing, by the physiologic sensor arrangement, physiologic sensor signals in response to the physiologic sensor measurement, measuring, using a non-physiologic sensor arrangement of the device, at least one non-physiologic parameter or at least one condition impacting a current context or wellbeing of the wearer, producing, by the non-physiologic sensor arrangement, non-physiologic sensor signals in response to the non-physiologic sensor measurement, and detecting, using a controller of the device, one or more of presence, absence, and severity of tinnitus of the wearer using the physiologic sensor signals and the non-physiologic sensor signals.

The above summary is not intended to describe each disclosed embodiment or every implementation of the present disclosure. The figures and the detailed description below more particularly exemplify illustrative embodiments.

### BRIEF DESCRIPTION OF THE DRAWINGS

Throughout the specification reference is made to the appended drawings wherein:

FIG. 1A illustrates an ear-wearable electronic device which incorporates an automatic tinnitus masker in accordance with any of the embodiments disclosed herein;

FIG. 1B illustrates a representative physiologic sensor arrangement, a non-physiologic sensor arrangement, and contextual factor data circuitry of the automatic tinnitus masker shown in FIG. 1A in accordance with any of the embodiments disclosed herein;

FIG. 2 illustrates a method of detecting tinnitus of a hearing device wearer in accordance with any of the embodiments disclosed herein;

FIG. 3 illustrates a method of detecting tinnitus of a hearing device wearer in accordance with any of the embodiments disclosed herein;

FIG. 4 illustrates a method of detecting tinnitus of a hearing device wearer in accordance with any of the embodiments disclosed herein;

FIG. 5 illustrates a method of detecting tinnitus of a hearing device wearer and automatically adjusting a tinnitus masking sound generated by the hearing device in accordance with any of the embodiments disclosed herein;

FIG. 6 illustrates a method of detecting tinnitus of a hearing device wearer and automatically adjusting a tinnitus masking sound generated by the hearing device in accordance with any of the embodiments disclosed herein;

FIG. 7 illustrates a method of detecting tinnitus of a hearing device wearer, automatically adjusting a masker function, and/or launching other applications in accordance with any of the embodiments disclosed herein;

FIGS. 8A and 8B illustrate aspects of machine learning by a machine learning processor of a hearing device configured to receive and operate on a wide variety of sensor data and contextual data in accordance with any of the embodiments disclosed herein;

FIG. 9 also shows various measures that can be implemented using a hearing device to mitigate tinnitus of the hearing device wearer in accordance with any of the embodiments disclosed herein;

FIGS. 9A and 9B show qualitative and quantitative data demonstrating that a significant improvement in a wearer's tinnitus can be achieved when treated using an automatic tinnitus masker function implemented in accordance with any of the embodiments disclosed herein; and

FIG. 10 is a block diagram of a representative ear-wearable electronic device which can incorporate a tinnitus detection and mitigation facility in accordance with any of the embodiments disclosed herein.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

Embodiments of the disclosure are directed to an ear-wearable electronic device which includes a sound generator and electronics configured to implement an automatic tinnitus masker function in accordance with any of the embodiments disclosed herein. Tinnitus is the chronic perception of phantom ringing in the ears which currently affects tens of millions of people in the United States alone. More particularly, tinnitus is an auditory phantom perception of chronic high-pitched sound, noise, or ringing, typically in the frequency range of 6-8 kHz, without any objective external sound source. Tinnitus is a common artifact of hearing loss, but may also be a symptom of other underlying conditions, such as ear injuries and circulatory system disorders.

Although tinnitus effects can range from mild to severe, a significant percentage of tinnitus sufferers describe their tinnitus as disabling or nearly disabling. Tinnitus is a distressing condition that can be mitigated via sound treatment. Sound treatment refers to playing “masker” sounds, often via an ear-wearable electronic device, to cover up individuals' tinnitus. Tinnitus maskers are most effective when their output level is set to where individuals can just barely hear their tinnitus. To more effectively provide relief to a person suffering from tinnitus, the output level of the sound masker should vary based on changes to tinnitus loudness and even be off when the tinnitus is not bothersome.

In accordance with any of the embodiments disclosed herein, an ear-wearable electronic device comprises an automatic tinnitus masker facility configured to one or more both of detect presence and absence of tinnitus of a device wearer using one or more of physiologic sensor signals, non-physiologic sensor signals, and contextual factor data indicative of one or more factors impacting a current context or wellbeing of the wearer. According to any of the embodiments disclosed herein, an ear-wearable electronic device comprises an automatic tinnitus masker facility configured to detect presence and severity of tinnitus of a device wearer using one or more of physiologic sensor signals, non-physiologic sensor signals, and contextual factor data indicative of one or more factors impacting a current context or wellbeing of the wearer. In accordance with any of the embodiments disclosed herein, an ear-wearable electronic device comprises an automatic tinnitus masker facility configured to adjust a tinnitus masking sound produced by the device using one or more of physiologic sensor signals, non-physiologic sensor signals, and contextual factor data indicative of one or more factors impacting a current context or wellbeing of the wearer.

The automatic tinnitus masker facility can be configured to generate a tinnitus masking sound that dynamically matches or substantially matches the tinnitus sound currently perceived by the hearing device wearer using one or more of physiologic sensor signals, non-physiologic sensor signals, and contextual factor data indicative of one or more factors impacting a current context or wellbeing of the wearer. The automatic tinnitus masker facility can be configured to generate a tinnitus masking sound that dynamically matches or substantially matches the tinnitus sound currently perceived by the hearing device wearer in terms of one or more of a loudness level, pitch, frequency bandwidth, and frequency composition.

According to any of the embodiments disclosed herein, an ear-wearable electronic device comprises an automatic tinnitus masker facility and a machine learning processor configured to receive one or more of physiologic sensor signals, non-physiologic sensor signals, and contextual factor data indicative of one or more factors impacting a current context or wellbeing of the wearer, and determine one or more of tinnitus presence, absence, and severity. This determination is then used by the device to drive changes in masker output level and/or to turn the masker on/off. The automatic tinnitus masker facility of the hearing device obviates the need for the wearer to apply frequent manual adjustments to the masker.

The term ear-wearable electronic device refers to a wide variety of electronic devices configured for deployment in, on or about an ear of a wearer. The term “ear-wearable electronic device” refers to a wide range of ear-level electronic devices, including hearing assistance devices, personal amplification devices, ear-protective devices and appliances, and other hearables (referred to herein generically or collectively as “hearing devices”).

Representative ear-wearable electronic devices of the present disclosure include, but are not limited to, in-the-canal (ITC), completely-in-the-canal (CIC), invisible-in-canal (IIC), in-the-ear (ITE), receiver-in-canal (RIC), behind-the-ear (BTE), and receiver-in-the-ear (RITE) type devices. Representative ear-wearable electronic devices of the present disclosure include, but are not limited to, earbuds, electronic ear plugs, personal sound amplification devices, and other ear-wearable electronic appliances. Ear-wearable electronic devices of the present disclosure include various types of hearing devices, various types of physiologic monitoring and biometric devices, and combined hearing/physiologic monitoring devices. Ear-wearable electronic devices of the present disclosure include restricted medical devices (e.g., devices regulated by the U.S. Food and Drug Administration), such as hearing aids and bone conduction hearing devices. Ear-wearable electronic devices of the present disclosure include consumer electronic devices, such as consumer earbuds, consumer sound amplifiers, and consumer hearing devices (e.g., consumer hearing aids and over-the-counter (OTC) hearing devices), for example.

Ear-wearable electronic devices according to any of the embodiments disclosed herein include an enclosure, such as a housing or shell, within which internal components are disposed. Typical components of an ear-wearable electronic device include a sound generator disposed in the housing and configured to produce at least a tinnitus masking sound, a physiologic sensor arrangement, and a controller operatively coupled to the sound generator and the physiologic sensor arrangement. The physiologic sensor arrangement is configured to measure a plurality of physiologic parameters and/or physiologic conditions of the wearer, and is configured to produce physiologic sensor signals in response to the measurements. The physiologic sensor arrangement is configured to measure and/or receive one or more of an electroencephalogram (EEG) signal, an electrocardiogram (ECG) signal, an electromyogram (EMG) signal, an electrooptigram (EOG), an electrodermal activity, a galvanic skin response (GSR) signal, a photoplethysmogram (PPG) signal, and biochemical sensor signal, for example. The controller is configured to detect one or more of presence, absence, and severity of tinnitus of the device wearer using at least the physiologic sensor signals. The device can be configured to automatically adjust the tinnitus masking sound produced by the sound generator using at least the physiologic sensor signals.

Ear-wearable electronic devices according to any of the embodiments disclosed herein include a sound generator disposed in a housing and configured to produce at least a tinnitus masking sound, a non-physiologic sensor arrangement, and a controller operatively coupled to the sound generator and the non-physiologic sensor arrangement. The non-physiologic sensor arrangement is configured to measure a plurality of non-physiologic parameters and/or conditions of the wearer (e.g., ambient light, ambient barometric pressure, ambient sound, wearer motion), and is configured to produce non-physiologic sensor signals in response to the measurements. The controller is configured to detect one or more of presence, absence, and severity of tinnitus of the device wearer using at least the non-physiologic sensor signals. The device can be configured to automatically adjust the tinnitus masking sound produced by the sound generator using at least the non-physiologic sensor signals.

Ear-wearable electronic devices according to any of the embodiments disclosed herein include a sound generator disposed in a housing and configured to produce at least a tinnitus masking sound, a communication device, and a controller operatively coupled to the sound generator and the communication device. The communication device is configured to receive, from an external electronic device or system, contextual factor data indicative of one or more factors impacting a current context or wellbeing of the wearer (e.g., time of day data, wearer sleep data, wearer nutrition data, local weather data, wearer stress data, and wearer medication data). The controller is configured to detect one or more of presence, absence, and severity of tinnitus of the device wearer using at least the contextual factor data. The device can be configured to automatically adjust the tinnitus masking sound produced by the sound generator using at least the contextual factor data.

Ear-wearable electronic devices according to any of the embodiments disclosed herein include a sound generator disposed in a housing and configured to produce at least a tinnitus masking sound, a physiologic sensor arrangement configured to produce physiologic sensor signals in response to at least one physiologic sensor measurement, a non-physiologic sensor arrangement configured to produce non-physiologic sensor signals in response to at least one non-physiologic sensor measurement, and a controller operatively coupled to the sound generator, the physiologic sensor arrangement, and the non-physiologic sensor arrangement. The controller is configured to detect one or more of presence, absence, and severity of tinnitus of the device wearer using at least the physiologic sensor signals and the non-physiologic sensor signals. The device can be configured to automatically adjust the tinnitus masking sound produced by the sound generator using at least the physiologic sensor signals and the non-physiologic sensor signals.

Ear-wearable electronic devices according to any of the embodiments disclosed herein include a sound generator disposed in a housing and configured to produce at least a tinnitus masking sound, a communication device configured to receive contextual factor data from an external electronic device or system, a physiologic sensor arrangement configured to produce physiologic sensor signals in response to at least one physiologic sensor measurement, a non-physiologic sensor arrangement configured to produce non-physiologic sensor signals in response to at least one non-physiologic sensor measurement, and a controller operatively coupled to the sound generator, the communication device, the physiologic sensor arrangement, and the non-physiologic sensor arrangement. The controller is configured to detect one or more of presence, absence, and severity of tinnitus of the device wearer using at least the physiologic sensor signals, the non-physiologic sensor signals, and the contextual factor data. The device can be configured to automatically adjust the tinnitus masking sound produced by the sound generator using at least the physiologic sensor signals, the non-physiologic sensor signals, and the contextual factor data.

Embodiments of the disclosure are defined in the claims. However, below there is provided a non-exhaustive listing of non-limiting examples. Any one or more of the features of these examples may be combined with any one or more features of another example, embodiment, or aspect described herein.

Example Ex1. An ear-wearable electronic device comprises a housing configured to be worn in, at or about an ear of a wearer, and a sound generator disposed in the housing and configured to produce at least a tinnitus masking sound. A physiologic sensor arrangement is disposed in or supported by the housing and configured to measure a plurality of physiologic parameters or physiologic conditions of the wearer, the physiologic sensor arrangement configured to produce physiologic sensor signals in response to the physiologic sensor measurements. A controller is operatively coupled to the sound generator and the physiologic sensor arrangement, the controller configured to detect one or more of presence, absence, and severity of tinnitus of the wearer using the physiologic sensor signals.

Example Ex2. An ear-wearable electronic device comprises a housing configured to be worn in, at or about an ear of a wearer, and a sound generator disposed in the housing and configured to produce at least a tinnitus masking sound. A physiologic sensor arrangement is disposed in or supported by the housing and configured to measure at least one physiologic parameter or at least one physiologic condition of the wearer, the physiologic sensor arrangement configured to produce physiologic sensor signals in response to the physiologic sensor measurement. A non-physiologic sensor arrangement comprises one or more non-physiologic sensors configured to sense a least one non-physiologic parameter or condition impacting a current context or wellbeing of the wearer, the non-physiologic sensor arrangement configured to produce non-physiologic sensor signals in response to the non-physiologic sensor measurement. A controller is operatively coupled to the sound generator, the physiologic sensor arrangement, and the non-physiologic sensor arrangement, the controller configured to detect one or more of presence, absence, and severity of tinnitus of the wearer using the physiologic sensor signals and the non-physiologic sensor signals.

Example Ex3. An ear-wearable electronic device comprises a housing configured to be worn in, at or about an ear of a wearer, and a sound generator disposed in the housing and configured to produce at least a tinnitus masking sound. A communication device is disposed in or supported by the housing and configured to wirelessly communicate with an external electronic device or system and to receive, from the external electronic device or system, contextual factor data indicative of one or more factors impacting a current context or wellbeing of the wearer. A physiologic sensor arrangement is disposed in or supported by the housing and configured to measure at least one physiologic parameter or at least one physiologic condition of the wearer, the physiologic sensor arrangement configured to produce physiologic sensor signals in response to the measurement. A non-physiologic sensor arrangement comprises one or more non-physiologic sensors configured to sense at least one non-physiologic parameter or condition impacting a current context or wellbeing of the wearer, the non-physiologic sensor arrangement configured to produce non-physiologic sensor signals in response to the non-physiologic sensor measurement. A controller is operatively coupled to the sound generator, the communication device, the physiologic sensor arrangement, and the non-physiologic sensor arrangement, the controller configured to detect one or more of presence, absence, and severity of tinnitus of the wearer using the physiologic sensor signals, the non-physiologic sensor signals, and the contextual factor data.

Example Ex4. The device according to one or more of Ex1 to Ex3, wherein the device is devoid of a microphone.

Example Ex5. The device according to one or more of Ex1 to Ex4, wherein the physiologic sensor arrangement comprises a sensor configured to produce an electroencephalography (EEG) signal.

Example Ex6. The device according to Ex5, wherein the controller is configured to produce EEG spectral power data using the EEG signal, detect one or more of absence, presence, and severity of tinnitus of the wearer using the EEG spectral power data.

Example Ex7. The device according to one or more of Ex1 to Ex6, wherein the physiologic sensor arrangement comprises a sensor configured to produce an electrocardiogram (ECG) signal.

Example Ex8. The device according to Ex7, wherein the controller is configured to produce heart rate variability data using the ECG signal, and detect one or more of absence, presence, and severity of tinnitus of the wearer using the heart rate variability data.

Example Ex9. The device according to one or more of Ex1 to Ex8, wherein the physiologic sensor arrangement comprises a sensor configured to produce a photoplethysmography (PPG) signal.

Example Ex10. The device according to Ex9, wherein the controller is configured to produce heart rate variability data using the PPG signal, and detect one or more of absence, presence, and severity of tinnitus of the wearer using the heart rate variability data.

Example Ex11. The device according to one or more of Ex1 to Ex10, wherein the physiologic sensor arrangement comprises a sensor configured to produce an electromyography (EMG) signal.

Example Ex12. The device according to one or more of Ex1 to Ex 11, wherein the physiologic sensor arrangement comprises a sensor configured to produce an electrooculography (EOG) signal.

Example Ex13. The device according to one or more of Ex1 to Ex12, wherein the physiologic sensor arrangement comprises a sensor configured to produce one or both of an electrodermal activity signal and a galvanic skin response signal.

Example Ex14. The device according to one or more of Ex1 to Ex13, wherein the physiologic sensor arrangement comprises a biochemical sensor.

Example Ex15. The device according to Ex14, wherein the biochemical sensor is configured to sense changes in blood serotonin levels of the wearer.

Example Ex16. The device according to Ex14, wherein the biochemical sensor is configured to sense changes in blood glucose levels of the wearer.

Example Ex17. The device according to one or more of Ex1 to Ex16, comprising a motion sensor disposed in or supported by the housing and coupled to the controller, the motion sensor configured to generate a motion sensor signal indicative of wearer motion, wherein the controller is configured to detect one or more of absence, presence, and severity of tinnitus of the wearer using the motion sensor signal.

Example Ex18. The device according to one or more of Ex1 to Ex17, comprising an optical sensor supported by the housing and coupled to the controller, the optical sensor configured to generate an optical sensor signal indicative of ambient light intensity, wherein the controller is configured to detect one or more of absence, presence, and severity of tinnitus of the wearer using the optical sensor signal.

Example Ex19. The device according to one or more of Ex1 to Ex18, comprising a microphone arrangement supported by the housing and coupled to the controller, the microphone arrangement comprising one or more microphones configured to generate a microphone signal indicative of sound within the wearer's current acoustic environment, wherein the controller is configured to detect one or more of absence, presence, and severity of tinnitus of the wearer using the microphone signal.

Example Ex20. The device according to one or more of Ex1 to Ex19, comprising a barometric pressure sensor supported by the housing and coupled to the controller, the barometric pressure sensor configured to generate a pressure sensor signal indicative of ambient barometric pressure, wherein the controller is configured to detect one or more of absence, presence, and severity of tinnitus of the wearer using the pressure sensor signal.

Example Ex21. The device according to one or more of Ex3 to Ex20, wherein the contextual factor data comprises one or more of time of day data, local weather data, wearer sleep data, data indicative of the wearer's nutrition, wearer stress data, wearer medication data.

Example Ex22. The device according to Ex21, wherein the data indicative of the wearer's nutrition comprises one or more of the wearer's caffeine intake, nicotine intake, salt intake, and alcohol intake.

Example Ex23. The device according to one or more of Ex1 to Ex22, wherein the controller is configured to adjust the tinnitus masking sound produced by the sound generator.

Example Ex24. The device according to one or more of Ex1 to Ex23, wherein the controller is configured to adjust the tinnitus masking sound produced by the sound generator by producing a tinnitus masking sound that substantially matches the wearer's tinnitus.

Example Ex25. The device according to one or more of Ex1 to Ex24, wherein the controller is configured to adjust the tinnitus masking sound produced by the sound generator using the physiologic sensor signals.

Example Ex26. The device according to Ex25, wherein the physiologic sensor signals are produced by two or more of an EEG sensor, an ECG sensor, an EMG sensor, an EOG sensor, a PPG sensor, an electrodermal activity sensor, a GSR sensor, and a biochemical sensor.

Example Ex27. The device according to one or more of Ex2 to Ex26, wherein the controller is configured to adjust the tinnitus masking sound produced by the sound generator using the physiologic sensor signals and the non-physiologic sensor signals.

Example Ex28. The device according to Ex27, wherein the controller is configured to adjust the tinnitus masking sound produced by the sound generator using the physiologic sensor signals and a signal produced by one or more of a motion sensor, an optical sensor, and a microphone.

Example Ex29. The device according to one or more of Ex3 to Ex28, wherein the controller is configured to adjust the tinnitus masking sound produced by the sound generator using the physiologic sensor signals, the non-physiologic sensor signals, and the contextual factor data.

Example Ex30. The device according to Ex29, wherein the contextual factor data comprises one or more of time of day data, wearer sleep data, wearer nutrition data, local weather data, wearer stress data, and wearer medication data.

Example Ex31. The device according to one or more of Ex1 to Ex30, wherein the controller is configured to adjust one or more of a loudness level, a bandwidth, a noise type, a pitch, a frequency composition, and a frequency shaping of the tinnitus masking sound produced by the sound generator.

Example Ex32. The device according to one or more of Ex1 to Ex31, comprising a microphone arrangement supported by the housing and coupled to the controller, the microphone arrangement comprising one or more microphones configured to generate a microphone signal indicative of sound within the wearer's current acoustic environment, wherein the controller is configured to classify the current acoustic environment of the wearer as a specified one of a plurality of disparate acoustic environments, adjust the tinnitus masking sound produced by the sound generator using one or more of the physiologic sensor signals, the non-physiologic sensor signals, and the contextual factor data, and parameter values associated with the specified acoustic environment.

Example Ex33. The device according to Ex33, wherein the controller is configured to adjust a frequency response of the tinnitus masker sound in frequency bands appropriate for the specified acoustic environment.

Example Ex34. The device according to one or more of Ex1 to Ex33, wherein the controller is configured to adjust, in response to first physiologic sensor signals, the tinnitus masking sound produced by the sound generator, and detect mitigation or non-mitigation of the wearer's tinnitus in response to second physiologic sensor signals acquired subsequent to the tinnitus masking sound adjustment.

Example Ex35. The device according to one or more of Ex1 to Ex34, wherein the controller is configured to adjust, in response to the physiologic sensor signals, the tinnitus masking sound produced by the sound generator, and override the tinnitus masking sound adjustment in response to an input received from the wearer.

Example Ex36. The device according to one or more of Ex1 to Ex37, wherein the controller comprises, or is operatively coupled to, a processor configured with instructions to process one or more of the physiologic sensor signals, the non-physiologic sensor signals, and the contextual factor data via a machine learning algorithm to detect one or more of presence, absence, and severity of tinnitus of the wearer.

Example Ex37. The device according to one or more of Ex1 to Ex36, wherein the controller comprises, or is operatively coupled to, a processor configured with instructions to process one or more of the physiologic sensor signals, the non-physiologic sensor signals, and the contextual factor data via a machine learning algorithm to adjust the tinnitus masking sound produced by the sound generator.

Example Ex38. The device according to one or more of Ex1 to Ex37, wherein the controller comprises, or is operatively coupled to, a processor configured with instructions to process, via a machine learning algorithm, the one or more of the physiologic sensor signals, the non-physiologic sensor signals, and the contextual factor data to adjust one or more of a loudness level, a bandwidth, a noise type, a pitch, a frequency composition, and a frequency shaping of the tinnitus masking sound produced by the sound generator.

Example Ex39. The device according to one or more of Ex1 to Ex38, comprising a microphone arrangement supported by the housing and coupled to the controller, the microphone arrangement comprising one or more microphones configured to generate a microphone signal indicative of sound within the wearer's current acoustic environment, wherein the controller comprises, or is operatively coupled to, a processor configured with instructions to classify, via a first neural network, the acoustic environment of the wearer as a specified one of a plurality of disparate acoustic environments, and process one or more of the physiologic sensor signals, the non-physiologic sensor signals, and the contextual factor data, via a second neural network, to adjust the tinnitus masking sound produced by the sound generator using the one or more of the physiologic sensor signals, the non-physiologic sensor signals, and the contextual factor data, and parameter values associated with the specified acoustic environment.

Example Ex40. The device according to Ex39, wherein the processor is configured with instructions to adjust a frequency response of the tinnitus masker sound in frequency bands appropriate for the specified acoustic environment.

Example Ex41. The device according to one or more of Ex1 to Ex40, wherein the controller comprises, or is operatively coupled to, a processor configured with instructions to process first data comprising one or more of the physiologic sensor signals, the non-physiologic sensor signals, and the contextual factor data via a machine learning algorithm to adjust the tinnitus masking sound produced by the sound generator, and detect mitigation or non-mitigation of the wearer's tinnitus, via the neural network, in response to second data comprising one or more of the physiologic sensor signals, the non-physiologic sensor signals, and the contextual factor data acquired subsequent to the tinnitus masking sound adjustment.

Example Ex42. The device according to one or more of Ex1 to Ex41, wherein the controller comprises, or is operatively coupled to, a processor configured with instructions to process first data comprising one or more of the physiologic sensor signals, the non-physiologic sensor signals, and the contextual factor data via a machine learning algorithm to adjust the tinnitus masking sound produced by the sound generator, and override the tinnitus masking sound adjustment in response to an input received from the wearer.

Example Ex43. The device according to one or more of Ex36 to Ex42, wherein the processor is configured to process data comprising one or more of the physiologic sensor signals, the non-physiologic sensor signals, and the contextual factor data via a neural network.

Example Ex44. The device according to Ex43, wherein the neural network comprises one or more of a deep neural network (DNN), a feedforward neural network (FNN), a recurrent neural network (RNN), a long short-term memory (LSTM), gated recurrent units (GRU), light gated recurrent units (LiGRU), a convolutional neural network (CNN), and a spiking neural network.

Example Ex45. The device according to one or more of Ex1 to Ex44, wherein the ear-wearable electronic device comprises an in-ear hearing device or an on-ear hearing device.

Example Ex46. The device according to one or more of Ex1 to Ex45, wherein the ear-wearable electronic device comprises a hearing aid.

Example Ex47. The device according to one or more of Ex1 to Ex45, wherein the ear-wearable electronic device comprises an electronic ear plug.

Example Ex48. A method implemented by an ear-wearable electronic device worn by a wearer comprises measuring, using a physiologic sensor arrangement of the device, a plurality of one or both of physiologic parameters and physiologic conditions of the wearer, producing, by the physiologic sensor arrangement, physiologic sensor signals in response to the physiologic sensor measurements, and detecting, using a controller of the device, one or more of presence, absence and severity of tinnitus of the wearer using the physiologic sensor signals.

Example Ex49. A method implemented by an ear-wearable electronic device worn by a wearer comprises measuring, using a physiologic sensor arrangement of the device, at least one physiologic parameter or at least one physiologic condition of the wearer, producing, by the physiologic sensor arrangement, physiologic sensor signals in response to the physiologic sensor measurement, measuring, using a non-physiologic sensor arrangement of the device, at least one non-physiologic parameter or at least one condition impacting a current context or wellbeing of the wearer, producing, by the non-physiologic sensor arrangement, non-physiologic sensor signals in response to the non-physiologic sensor measurement, and detecting, using a controller of the device, one or more of presence, absence, and severity of tinnitus of the wearer using the physiologic sensor signals and the non-physiologic sensor signals.

Example Ex50. A method implemented by an ear-wearable electronic device worn by a wearer comprises measuring, using a physiologic sensor arrangement of the device, at least one physiologic parameter and or at least one physiologic condition of the wearer, producing, by the physiologic sensor arrangement, physiologic sensor signals in response to the physiologic sensor measurement, measuring, using a non-physiologic sensor arrangement of the device, at least one non-physiologic parameter or at least one condition impacting a current context or wellbeing of the wearer, producing, by the non-physiologic sensor arrangement, non-physiologic sensor signals in response to the non-physiologic sensor measurement, and detecting, using a controller of the device, one or more of presence, absence, and severity of tinnitus of the wearer using the physiologic sensor signals and the non-physiologic sensor signals.

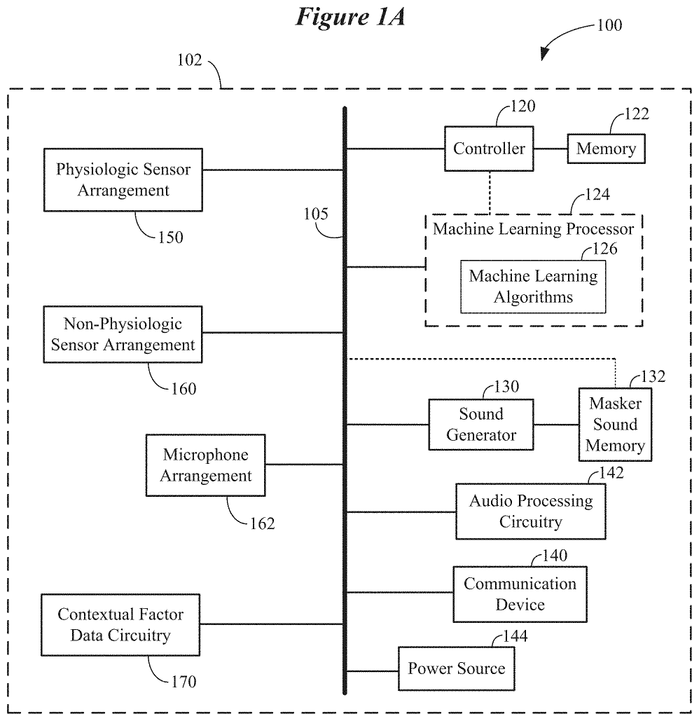

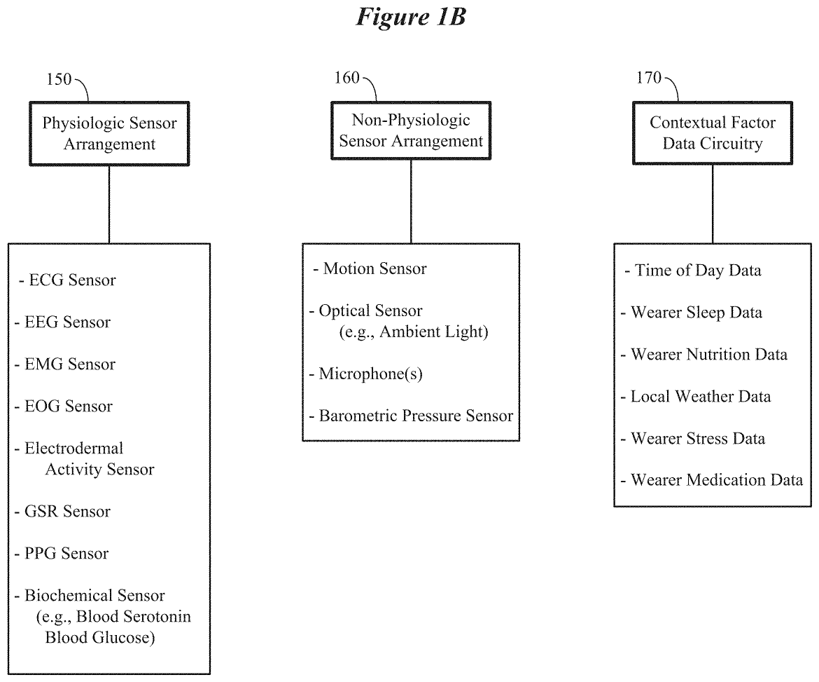

FIG. 1A illustrates an ear-wearable electronic device in accordance with any of the embodiments disclosed herein. The ear-wearable electronic device 100, referred to hereinbelow as hearing device 100 for convenience, includes a housing 102 configured for deployment in, on or about an ear of a wearer. According to any of the embodiments disclosed herein, the housing 102 can be configured for deployment at least partially within the wearer's ear. For example, the housing 102 can be configured for deployment at least partially or entirely within an ear canal of the wearer's ear. In some configurations, the shape of the housing 102 can be customized for the wearer's ear canal (e.g., based on a mold taken from the wearer's ear canal). In other configurations, the housing 102 can be constructed from pliant (e.g., semisoft) material that, when inserted into the wearer's ear canal, takes on the shape of the ear canal.

According to any of the embodiments disclosed herein, the housing 102 can be configured for deployment at least partially within the outer ear, such as from the helix to the ear canal (e.g., the concha cymba, concha cavum) and can extend up to or into the ear canal. According to any of the embodiments disclosed herein, the housing 102 can be configured for deployment at or on the wearer's outer ear (e.g., auricle or pinna), such as behind the wearer's ear or situated on or over (but in contact with) the wearer's ear without extending into the wearer's ear or ear canal.

The housing 102 of the hearing device 100 is configured to contain or support a number of components including a controller 120 which includes, or is coupled to, memory 122, and a power source 144 (e.g., rechargeable battery, power management circuitry, power conversion circuitry). The controller 120 is operatively coupled to each of the components shown in FIG. 1A via communication bus 105 (e.g., a rigid or flexile PCB), it being understood that some of the components can be coupled to the controller 120 via a direct connection. A sound generator 130 is disposed in the housing 102 and configured to produce at least a tinnitus masking sound. In some implementations, the sound generator 130 is configured to serve as conventional speaker or receiver (e.g., a hearing aid speaker or receiver), in addition to generating a tinnitus masking sound.

The sound generator 130 can include, or be coupled to (e.g., directly or indirectly) a masker sound memory 132. The masker sound memory 132 is configured to store sound data indicative of one or more masker sounds that can help the auditory system become less sensitive to tinnitus and may also promote relaxation by reducing the contrast between tinnitus sounds and background sound. The masker sound memory 132 can be configured to store sound data indicative of one or more synthetic masker sounds, such as pink noise, brown noise, and white noise. The masker sound memory 132 can be configured to store data indicative of one or more natural masker sounds, such as ocean surf and rainfall. The masker sound memory 132 can also be configured to store data indicative of one or more tinnitus therapy sounds, such as binaural beats or ocean wave sounds.

In accordance with any of the embodiments disclosed herein, a physiologic sensor arrangement 150 is disposed in, or supported by, the housing 102 and configured to measure one or both of a plurality of physiologic parameters and a plurality of physiologic conditions of the wearer. The physiologic sensor arrangement 150, operatively coupled to the controller 120, is configured to produce physiologic sensor signals in response to measurements made by one or more physiologic sensors of the arrangement 150. Examples of various physiologic sensors that can be included in the physiologic sensor arrangement 150 are shown in FIG. 1B.

In accordance with any of the embodiments disclosed herein, a non-physiologic sensor arrangement 160 is disposed in, or supported by, the housing 102 and configured to measure one or both of a plurality of non-physiologic parameters and a plurality of non-physiologic conditions impacting the current context or wellbeing of the wearer. The non-physiologic sensor arrangement 160, operatively coupled to the controller 120, is configured to produce non-physiologic sensor signals in response to measurements made by one or more non-physiologic sensors of the arrangement 160. Examples of various non-physiologic sensors that can be included in the non-physiologic sensor arrangement 160 are shown in FIG. 1B.

In accordance with any of the embodiments disclosed herein, a communication device 140 and contextual factor data circuitry 170 can be included in, or supported by, the housing 102 and operatively coupled to the controller 120. The communication device 140 is configured to communicatively couple the hearing device 100 with one or more external electronic devices or systems (e.g., a smartphone, a tablet, a laptop, a cloud server, an Internet server). The communication device 140 is configured to receive, from the external electronic device or system, contextual factor data indicative of one or more factors impacting the current context or wellbeing of the wearer. The contextual factor data circuitry 170 is configured to operate on the received contextual factor data and communicate the contextual factor data in a form suitable for reception by the controller 120. Examples of various contextual factor data that can be received by the controller 120 via the communication device 140 are shown in FIG. 1B.

In accordance with any of the embodiments disclosed herein, the device 100 can be configured as a hearing device which includes an audio processing facility. The audio processing facility can include audio signal processing circuitry 142 coupled to the sound generator 130. The sound generator 130 includes an acoustic transducer, such as a speaker, a receiver, or a bone conduction transducer. In some configurations, the audio processing facility includes, or is coupled to, an arrangement of one or more microphones 162. In other configurations, the audio processing facility (and, therefore, the hearing device 100) is devoid of a microphone arrangement 162.

In accordance with any of the embodiments disclosed herein, the controller 120 can include, or be coupled to, a machine learning processor 124 configured to execute computer code or instructions (e.g., firmware, software) including one or more machine learning algorithms 126. The machine learning processor 124 is configured to process one or more of the physiologic sensor signals, non-physiologic sensor signals, microphone signals, and contextual factor data via one or more machine learning algorithms 126 to detect one or more of presence, absence, and severity of tinnitus of the wearer of the hearing device 100. Sensor, contextual factor data, and/or wearer input (e.g., manual overrides) received by the machine learning processor 124 are used to inform and refine one or more machine learning algorithms 126 executable by the machine learning processor 124 to automatically enhance and customize tinnitus detection and mitigation implemented by the hearing device 100 for a particular hearing device wearer.

For example, the machine learning processor 124 can be configured with executable instructions to process various sensor signals and/or contextual factor data via a machine learning algorithm 126 to detect one or more of presence, absence, and severity of tinnitus of the wearer. The machine learning processor 124 can be configured with executable instructions to process various sensor signals and/or contextual factor data via a machine learning algorithm 126 to adjust a masking sound delivered to the wearer's ear various sensor signals and/or contextual factor data to mitigate tinnitus of the wearer. The machine learning processor 124 can be configured with executable instructions to process one or more of physiologic sensor signals, non-physiologic sensor signals, and contextual factor data via a neural network and/or a support vector machine (SVM). The neural network can comprise one or more of a deep neural network (DNN), a feedforward neural network (FNN), a recurrent neural network (RNN), a long short-term memory (LSTM), gated recurrent units (GRU), light gated recurrent units (LiGRU), a convolutional neural network (CNN), and a spiking neural network.

According to any of the embodiments disclosed herein, the hearing device 100 can include one or more of a physiologic sensor arrangement 150, a non-physiologic sensor arrangement 160, and contextual factor data circuitry 170, representative examples of which are shown in FIG. 1B. The physiologic sensor arrangement 150 can include one or any combination of one or more bioelectric sensors, one or more optical physiologic sensors, and one or more biochemical sensors. A bioelectric sensor (e.g., electrode-based sensor) of the physiologic sensor arrangement 150 can include one or more of an impedance, conductance, resistance, and capacitance sensor.

The bioelectric sensors and sensing circuitry of the physiologic sensor arrangement 150 can be configured to measure and monitor electrical activity of various organs of the body, including the wearer's brain activity, muscle or nerve action potential (via an EEG sensor), heart (via an ECG sensor), musculature (via an EMG sensor), vision system (via an EOG sensor), and skin (via a GSR sensor and/or an electrodermal activity sensor). In some implementations, the physiologic sensor arrangement 150 can include a bed of microneedles or microelectrodes, subsets of which can be used as electrodes for sensing one or more of EEG, ECG, EMG, EOG, GSR, and electrodermal activity waveforms, as well as other sensing applications that benefit from intimate body fluid contact. In response to signals received from the bioelectric sensors, the bioelectric sensing circuitry produces a bioelectric sensor output signal which can be communicated to the controller 120.

An EEG sensor of the physiologic sensor arrangement 150 can be configured to measure and monitor neural activity of the wearer's brain (e.g., electrical activity of the brain, brain waves). For example, an EEG sensor can be configured to measure and monitor neural activity of the wearer's auditory system (e.g., the auditory cortex). An EEG sensor can be configured to detect spontaneous cortical activity and cortical activity modulated by tinnitus-matched sound delivered by the hearing device 100 to wearers suffering from tinnitus. EEG sensor signals can be used by the controller 120 of the hearing device 100 to detect one or more of presence, absence, and severity of tinnitus of the device wearer using EEG sensor signals, and to adjust a tinnitus masking sound produced by the hearing device 100 to mitigate the wearer's tinnitus using EEG sensor signals.

EEG activity differs significantly between healthy subjects and subjects with tinnitus. Several research groups have demonstrated that tinnitus correlates with altered EEG profiles with respect to different brain wave frequency bands. The controller 120 or other processor of the hearing device 100 (e.g., the machine learning processor 124) can be configured to analyze various frequency bands of brain waves known to be impacted by tinnitus (e.g., spontaneous tinnitus). The controller 120 can be configured to analyze EEG spectral power for the following classical frequency bands: 0.5-4 Hz (Delta (δ)), 4-8 Hz (Theta (θ)), 8-12 Hz (Alpha (α)), 12-30 Hz (Beta ((β)), and 30-150 Hz (Gamma (γ) (low Gamma (30-70 Hz) and high Gamma (70-150 Hz)), in 1 Hz bins, for example. Each of these frequency bands is associated with a different brain state. The Delta (δ) band is associated with sleep. The Theta (θ) band is associated with deep relaxation and inward focus. The Alpha (α) band is associated with high relaxation and passive attention. The Beta (β) band is anxiety dominant, and is associated with activity, external attention and relaxation. The Gamma (γ) band is associated with concentration.

The controller 120 or other processor of the hearing device 100 can be configured to detect presence, absence, and/or severity of tinnitus of the hearing device wearer relative to baseline EEG spectral power data established for the wearer or for a population. For example, the wearer of the hearing device 100 can be subject to a clinical evaluation during which baseline EEG spectral power data is acquired for each of the five brain states discussed above. The baseline EEG spectral power data can be acquired using an EEG sensor of the hearing device 100 and fitting software or by use of a multiple channel EEG head-worn (scalp) sensor arrangement (e.g., 64 to 256 electrodes). The baseline EEG spectral power data for a particular wearer can be transferred to, and stored in, the memory 122 of the hearing device 100.

During operation of the hearing device 100, EEG data can be acquired using an EEG sensor of the physiologic sensor arrangement 150. The controller 120 or other processor can be configured to compute spectral power data using a fast Fourier transform (FFT) applied to the acquired EEG sensor data. The FFT can be implemented by the controller 120 or other processor of the hearing device 100 to calculate a power spectrum of each of the five frequency bands. The controller 120 or other processor can detect presence, absence, and/or severity of the wearer's tinnitus by comparing the baseline EEG spectral power data to the acquired EEG spectral power data for each frequency band relative to one or more thresholds stored in the hearing device memory 122 (e.g., one or more EEG spectral power thresholds stored in the hearing device memory 122 established for the wearer when not experiencing tinnitus or for an equivalent demographic population).

For example, several research groups have demonstrated that tinnitus correlates with altered EEG spectral power data including a reduction of Alpha power, an increase of Delta power, an increase of Theta power, an increase of Beta power, and an increase in Gamma power. Other studies have demonstrated similar correlations between tinnitus and altered EEG spectral power data, with some differences from the correlations listed above. The hearing device controller 120 or other processor can be configured to compare the acquired EEG spectral power data to the baseline EEG spectral power data for some or all of the five frequency bands. The controller 120 or other processor can be configured to detect presence of the wearer's tinnitus in response to detecting a clinically significant difference in the spectral power data comparisons relative to detection thresholds established for some or all of the five frequency bands and stored in the hearing device memory 122. The detection thresholds can be representative of a specified standard deviation from baseline spectral power values, a specified percentage deviation from baseline spectral power values, or a specified magnitude deviation from baseline spectral power values. The controller 120 or other processor can be configured to detect absence of the wearer's tinnitus in response to detecting no clinically significant difference or only a negligible difference (e.g., <2-5%) in the spectral power data comparisons relative to the detection thresholds established for some or all of the five frequency bands.

The controller 120 or other processor can be configured to determine severity of the wearer's tinnitus in response to detecting a clinically significant difference in the spectral power data comparisons relative to severity thresholds established for some or all of the five frequency bands, and determining the magnitude of the deviation of the detected difference relative to the severity thresholds. The severity thresholds can be representative of a specified standard deviation from baseline spectral power values, a specified percentage deviation from baseline spectral power values, or a specified magnitude deviation from baseline spectral power values. The severity thresholds can be stored in the hearing device memory 122.

In accordance with some implementations, an EEG marker of tinnitus which can be used for detecting presence, absence, and/or severity of a wearer's tinnitus. The EEG marker comprises a replica of a wearer's tinnitus sound experience and a measure of the wearer's EEG response to increasing intensity of the sound. The replica of sound can be constructed from a wearer's subjective determination of sound most closely related to the annoying sound experienced by the wearer, and the wearer's EEG response can be measured as an EEG marker of the wearer's tinnitus. The controller 120 or other processor can be configured to detect presence, absence, and/or severity of the wearer's tinnitus in response to identifying the presence or absence of the EEG marker in the EEG data acquired by the EEG sensor of the physiologic sensor arrangement 150.

The EEG sensor can include a multiplicity of electrodes, such as a bed of electrodes, for acquiring a multiplicity of EEG sensor signals from the hearing device wearer. Additional details of these and other tinnitus detection features and functionality that can be incorporated in a hearing device 100 in accordance with any of the embodiments disclosed herein are described in U.S. Pat. No. 7,981,047 and in U.S. Published Patent Application No. 2012/0203130, both of which are incorporated herein by reference in their respective entireties.

An ECG sensor of the physiologic sensor arrangement 150 can be configured to measure and monitor cardiac activity and various parameters of same, such as heart rate and heart rate variability (HRV). Alternatively or in addition, a photoplethysmogram (PPG) sensor of the physiologic sensor arrangement 150 can be configured to measure and monitor cardiac activity and various parameters of same, such as heart rate and HRV (see discussion below). HRV data acquired by the ECG and/or PPG sensor of the physiologic sensor arrangement 150 can be processed by the controller 120 or other processor of the hearing device using frequency-domain techniques or time-domain techniques.

The HRV spectrum contains three major components: a very low frequency (VLF, below 0.04 Hz), a low frequency (LF, 0.04-0.15 Hz), and a high frequency (HF, 0.15-0.4 Hz). Hypertension and factors that increase blood pressure, such as stress, alcohol and caffeine, can make tinnitus more pronounced. In tinnitus subjects, the power of the high frequency component (HF) and total power (TP) of the HRV significantly decreases, and the low frequency (LF) to high frequency (HF) ratio (LF/HF) significantly increases. Typically, a long duration of tinnitus corresponds to a larger observable HRV change.

The controller 120 or other processor can be configured to detect presence, absence, and/or severity of the wearer's tinnitus in response to detecting clinically significant differences (and magnitude of these differences) between normal HRV parameters (e.g., HRV parameters associated with a population having the same or similar demographics as the hearing device wearer or HRV parameters of the wearer when not experiencing tinnitus) and HRV parameters developed from ECG data acquired from the wearer using the ECG sensor of the physiologic sensor arrangement 150. According to some studies, typical HRV parameters for healthy subjects include the following: TP (ms²)≈7.49, VLF (ms²)≈6.37, LF (ms²)≈5.98, HF (ms2)≈6.41, and LF/HF≈0.94. According to such studies, typical HRV parameters for subjects experiencing tinnitus include the following: TP (ms²)≈6.92, VLF (ms²)≈6.09, LF (ms²)≈5.54, HF (ms²)≈5.13, and LF/HF≈2.48.

The controller 120 or other processor can be configured to detect presence, absence, and/or severity of the wearer's tinnitus in response to a clinically significant difference between the wearer's LF/HF ratio and a threshold, such as 2.48 (e.g., >10-25% of the threshold). An LF/HF ratio threshold and/or other HRV parameter threshold can be established for the wearer when not experiencing tinnitus or for an equivalent demographic population. These thresholds can and be stored in the hearing device memory 122. The magnitude of the LF/HF ratio difference can be calculated by the controller 120 or other processor, which can correspond to different levels of tinnitus severity when compared against different severity thresholds corresponding to each of the different levels of tinnitus severity. The thresholds can be established in a manner previously described and stored in the hearing device memory 122.

The controller 120 or other processor can be configured to detect presence, absence, and/or severity of the wearer's tinnitus in response to a clinically significant difference between the wearer's time-domain HRV parameters and baseline time-domain HRV parameters. The baseline time-domain HRV parameters can be established for the wearer when not experiencing tinnitus or established based on a population having the same or similar demographics as the hearing device wearer. The time-domain HRV parameters detected and processed by the controller 120 or other process of the hearing device 100 can include one or more of the following: standard deviation of NN intervals (SDNN in ms), root mean square of successive RR interval differences (RMS SD in ms), and interbeat interval (MI in ms). Studies have shown that representative short-term normal values (e.g., for healthy subjects) for these HRV parameters are as follows:

| | | |
|---|---|---|
| SDNN (ms): | Mean (SD) 50 (16) | Range 32-93 |
| RMSSD (ms): | Mean (SD) 42 (15) | Range 19-75 |
| IBI (ms): | Mean (SD) 926 (90) | Range 785-1,160 |

The controller 120 or other processor of the hearing device 100 can be configured to detect presence, absence, and/or severity of the wearer's tinnitus in response to detecting a clinically significant difference in one or more of these time-domain HRV parameters relative to respective detection and severity thresholds. These detection and severity thresholds can be based on the short-term normal values listed above. The detection thresholds and severity thresholds can be representative of a specified standard deviation from the baseline time-domain HRV parameter values, a specified percentage deviation from the baseline time-domain HRV parameter values, or a specified magnitude deviation from the baseline time-domain HRV parameter values.

The physiologic sensor arrangement 150 can include a photoplethysmogram (PPG) sensor. The controller 120 or other processor can be configured to detect presence, absence, and/or severity of the wearer's tinnitus in response to detecting clinically significant differences (and magnitude of these differences) between the wearer's PPG sensor signals acquired by the PPG sensor and normal PPG sensor signals (e.g., one or more PPG signal thresholds stored in the hearing device memory 122 established for the wearer when not experiencing tinnitus or for an equivalent demographic population). The PPG sensor utilizes a light emitter (e.g., one or more LEDs or laser diodes) configured to couple light into skin of the wearer and a light detector (e.g., a photosensor or photon detector) configured to receive light from the skin resulting from the light produced by the light emitter. Optical sensing circuitry can be configured to produce a photoplethysmographic signal in response to light received by the light detector. In some configurations in which at least two light sources of the light emitter of different wavelengths are included in the PPG sensor, the optical sensing circuitry can be configured to produce a pulse oximetry signal in response to light received by the light detector. In response to signals received from the light detector, the optical sensing circuitry produces an optical sensor output signal which can be communicated to the controller 120.

The PPG waveform produced by the PPG sensor of the physiologic sensor arrangement 150 comprises a pulsatile (‘AC’) physiological waveform attributed to cardiac synchronous changes in the blood volume with each heartbeat, and is superimposed on a slowly varying (‘DC’) baseline with various lower frequency components attributed to respiration, sympathetic nervous system activity and thermoregulation. The PPG sensor output signal produced by the optical sensing circuitry can be used to perform a number of different physiologic measurements, such as measuring blood oxygen saturation, blood pressure, cardiac output, assessing autonomic function, and detecting peripheral vascular disease. By way of example, the PPG sensor output signal produced by the optical sensing circuitry can be used for measuring and monitoring one or more of heart rate, respiration, vascular condition/disease, arterial condition/disease, compliance and ageing, venous condition/disease, compliance and ageing, endothelial function, microvascular blood flow, vasospastic conditions, autonomic function monitoring, vasomotor function and thermoregulation, HRV, orthostasis, and other cardiovascular variability conditions. A PPG sensor can be configured to detect turbulent blood flow which can lead to tinnitus. The PPG sensor can be configured to detect irregular blood flow (e.g., turbulence) due to narrowing or kinking in a neck artery (carotid artery) or vein in the neck (jugular vein) leading to tinnitus.

The biochemical sensor(s) of the physiologic sensor arrangement 150 can be implemented as one or more devices capable of converting a chemical or biological quantity into an electrical signal. The biochemical sensor(s) can be configured to interact with one of a variety of body fluids, such as blood, sweat, and interstitial fluids. In various implementations, the biochemical sensor(s) include an analyte molecule, a chemically sensitive layer, and a transducer. In some implementations, the biochemical sensor(s) can include a bed of microneedles.

The biochemical sensor(s) can be configured to sense an ingredient and concentration of one of more body fluids of the skin. For example, the biochemical sensor(s) 115 can be configured to detect one or more of a blood serotonin level, a blood glucose concentration, a PH value, and a Ca+ concentration, for example. Biochemical sensing circuitry of the physiologic sensor arrangement 150 is configured to produce a biochemical sensor output signal using signals produced by the biochemical sensor(s), which are communicated to the controller 120 or other processor of the hearing device 100.

A biochemical sensor of the physiologic sensor arrangement 150 can be configured to measure a wearer's blood glucose level. Prolonged high blood sugar (e.g., unmanaged diabetes) can damage the eighth cranial nerve. As this nerve is responsible for conveying balance and sound input to the inner ear, which relays to the brain, damage to the eighth cranial nerve can cause tinnitus. The physiologic sensor arrangement 150 of the hearing device 100 can include a blood glucose sensor or meter configured to periodically measure a blood glucose level in any compact non-invasive manner known in the art and to generate a corresponding glucose level signal.

For example, blood glucose can be monitored via iontophoresis at the surface of the skin of the ear combined with enzyme detection. It is noted that an optical sensor of the physiologic sensor arrangement 150 can be configured to monitor glucose from the tympanic membrane by monitoring optical reflection and optical fluorescence from the tympanic membrane in response to IR and blue light. A biochemical sensor of the hearing device 100 configured to measure a wearer's blood glucose level can be implemented to include components, circuitry, and processes disclosed in U.S. Pat. No. 9,769,577 and U.S. Published Patent Application Nos. 2018/0220902 and 2011/0137141, which are incorporated herein by reference.

A biochemical sensor of the physiologic sensor arrangement 150 can be configured to measure a wearer's blood serotonin level. Elevated serotonin levels can cause certain nerve cells in the brain to become hyperactive when exposed to increased levels of serotonin. This elevation in serotonin levels can raise anxiety levels and can result in tinnitus. A biochemical sensor of the hearing device 100 configured to measure a wearer's blood serotonin level can be implemented to include components, circuitry, and processes disclosed in U.S. Published Patent Application No. 2018/0266979, which is incorporated herein by reference.

The biochemical sensor output signals can be communicated to the controller 120 or other processor of the hearing device (e.g., machine learning processor 124). The controller 120 or other processor can be configured to detect presence, absence, and/or severity of the wearer's tinnitus in response to detecting clinically significant differences (and magnitude of these differences) between the wearer's biochemical sensor output signals acquired by one or more biochemical sensors of the physiologic sensor arrangement 150 and normal biochemical sensor signals (e.g., one or more biochemical sensor signal thresholds stored in the hearing device memory 122 established for the wearer when not experiencing tinnitus or for an equivalent demographic population).

The non-physiologic sensor arrangement 160 can include one or more sensors configured to produce non-physiologic sensor signals which, together with physiologic sensor signals produced by the physiologic sensor arrangement 150, can be used by the controller 120 or other processor of the hearing device 100 to detect presence, absence, and/or severity of the wearer's tinnitus. Representative non-physiologic sensors include, but are not limited to, a motion sensor (e.g., an inertial measurement unit or IMU, accelerometer, gyroscope, magnetometer), an optical sensor configured to sense ambient light, one or more microphones (e.g., a fixed or directional microphone array), and an ambient barometric pressure sensor. The ambient barometric pressure sensor can be configured to sense rapid changes in air pressure (e.g., from flying, diving, or being close to an explosion) that can stress the eardrum and inner ear and cause hearing changes, along with tinnitus. In some implementations, one or more temperature sensors can be included in the non-physiologic sensor arrangement 160 to provide additional information concerning the hearing device wearer's activity level/wellbeing and/or ambient temperature. Suitable temperature sensors include thermistors, thermocouples, and resistance temperature detectors.

Contextual factor data circuitry 170 of the hearing device 100 is configured to receive contextual factor data from one or more external electronic devices or systems (e.g., a smartphone, a tablet, a laptop, a cloud server, an Internet server) via a communication device 140 of the hearing device 100. The contextual factor data comprises data indicative of one or more factors impacting the current context or wellbeing of the wearer. The contextual factor data circuitry 170 is configured to operate on the received contextual factor data and communicate the contextual factor data in a form suitable for reception by the controller 120 or other processor of the hearing device 100.

Representative examples of various contextual factor data that can be received by the controller 120 via the communication device 140 include time of day data (e.g., tinnitus can be perceived as louder and more distressing during the night and early morning hours from 12 AM to 8 AM), wearer sleep data (e.g., insomnia and insufficient sleep can worsen symptoms of tinnitus), wearer nutrition data (e.g., caffeine, salt, and alcohol intake can worsen symptoms of tinnitus), local weather data (e.g., changes in barometric pressure can worsen or lessen symptoms of tinnitus), wearer stress data (e.g., tinnitus worsens during and after stressful situations; stress can be detected using one or more of the physiologic sensors disclosed herein), and wearer medication data (e.g., medications known to cause/worsen tinnitus include: antibiotics such as polymyxin B, erythromycin, vancomycin and neomycin; cancer medications such as methotrexate and cisplatin; and diuretics such as bumetanide, ethacrynic acid, and furosemide). The representative contextual factor data represents factors known to impact (negatively or positively) a wearer's tinnitus.

In accordance with any of the embodiments disclosed herein, and as previously described, the data acquired by one or more of the physiologic sensor arrangement 150, non-physiologic sensor arrangement 160, and contextual factor data circuitry 170 can be input to a machine learning algorithm 126 implemented by a machine learning processor 124 of the hearing device 100. The machine learning algorithm 126 can be trained to distinguish the absence/presence of the wearer's tinnitus and the severity thereof using these data. The machine learning algorithm 126 can be refined using physiological sensor feedback and/or manual overrides from the user, as described herein.

Once the machine learning algorithm 126 has determined the presence/severity of the wearer's tinnitus, this determination can be used to trigger the onset of a tinnitus masker from the sound generator 130 in response to data indicative of high tinnitus loudness percept. Conversely, data indicative of no/minimal/reduced tinnitus loudness percept will trigger an offset of the tinnitus masker produced by the sound generator 130. In some implementations, changes in physiological activity of the wearer can be used to apply automatic output level adjustments to the tinnitus masker, such that the masker level is increased when data indicates that tinnitus percept is louder and vice versa. According to some implementations, a clinician can have control over activating/de-activating this feature in the fitting software. In an alternative embodiment, the hearing device wearer can control the activation/de-activation of this feature via buttons on the hearing device 100, a remote control accessory, or a smartphone application. Furthermore, the sensitivity of this feature can be varied (e.g., high sensitivity means that changes in physiological activity cause faster/larger automated changes in tinnitus masker level) either by the clinician or the hearing device wearer. These and other automatic features provided by the hearing device 100 provide for improved satisfaction among hearing device wearers with tinnitus by requiring fewer manual adjustments to their tinnitus masker.

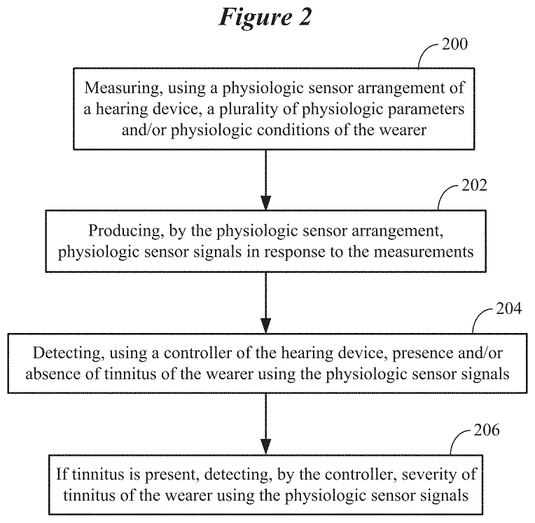

FIG. 2 illustrates a method of detecting tinnitus of a hearing device wearer in accordance with any of the embodiments disclosed herein. The method shown in FIG. 2 involves measuring 200, using a physiologic sensor arrangement of the hearing device, a plurality of physiologic parameters and/or physiologic conditions of the wearer. The method involves producing 202, by the physiologic sensor arrangement, physiologic sensor signals in response to the measurements. The method also involves detecting 204, using a controller (or processor) of the hearing device, presence and/or absence of tinnitus of the wearer using the physiologic sensor signals. The method further involves detecting 206, by the controller, severity of tinnitus of the wearer using the physiologic sensor signals if tinnitus is present.

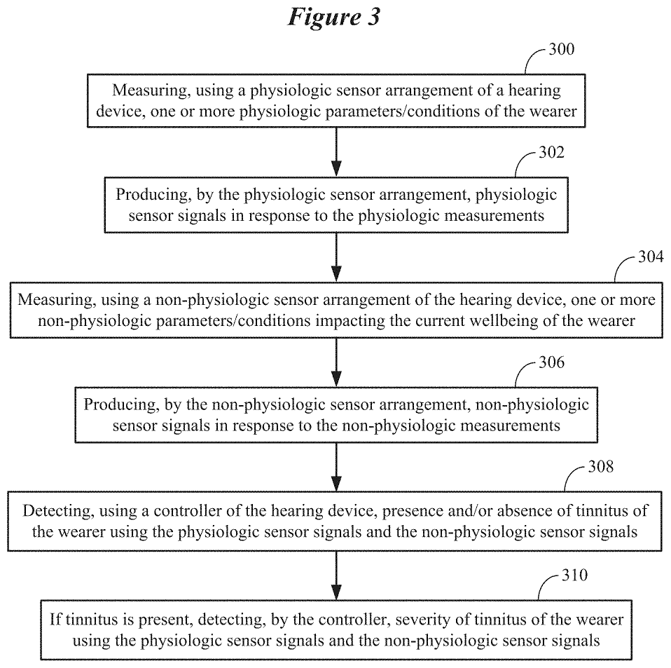

FIG. 3 illustrates a method of detecting tinnitus of a hearing device wearer in accordance with any of the embodiments disclosed herein. The method shown in FIG. 3 involves measuring 300, using a physiologic sensor arrangement of the hearing device, one or more physiologic parameters/physiologic conditions of the wearer, and producing 302, by the physiologic sensor arrangement, physiologic sensor signals in response to the physiologic measurements. The method also involves measuring 304, using a non-physiologic sensor arrangement of the hearing device, one or more non-physiologic parameters/physiologic conditions impacting the current context or wellbeing of the wearer, and producing 306, by the non-physiologic sensor arrangement, non-physiologic sensor signals in response to the non-physiologic measurements. The method shown in FIG. 3 further involves detecting 308, using a controller (or processor) of the hearing device, presence and/or absence of tinnitus of the wearer using the physiologic sensor signals and the non-physiologic sensor signals. The method also involves detecting 310, by the controller, severity of tinnitus of the wearer using the physiologic sensor signals and the non-physiologic sensor signals if tinnitus is present.

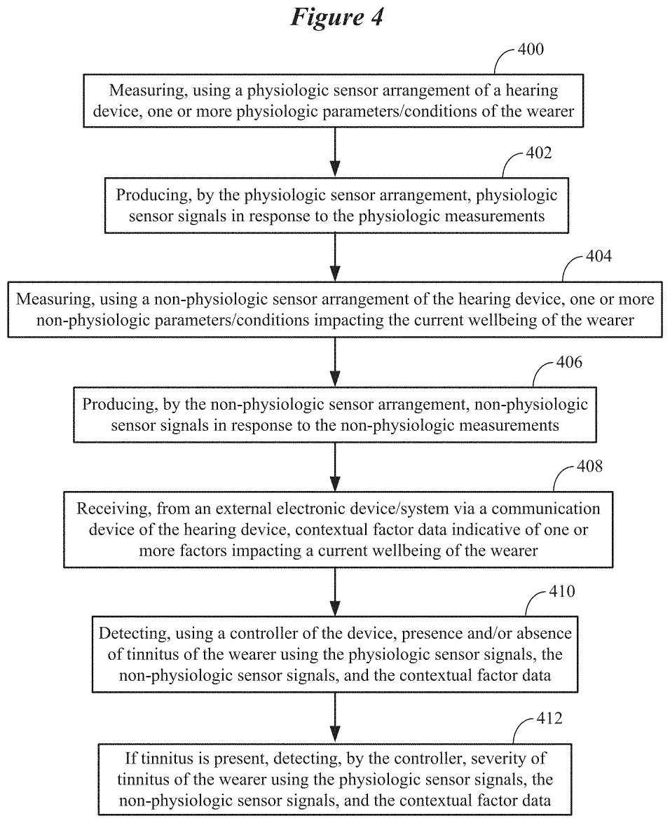

FIG. 4 illustrates a method of detecting tinnitus of a hearing device wearer in accordance with any of the embodiments disclosed herein. The method shown in FIG. 4 involves measuring 400, using a physiologic sensor arrangement of the hearing device, one or more physiologic parameters/physiologic conditions of the wearer, and producing 402, by the physiologic sensor arrangement, physiologic sensor signals in response to the physiologic measurements. The method also involves measuring 404, using a non-physiologic sensor arrangement of the hearing device, one or more non-physiologic parameters/physiologic conditions impacting the current context or wellbeing of the wearer, and producing 406, by the non-physiologic sensor arrangement, non-physiologic sensor signals in response to the non-physiologic measurements. The method further involves receiving 408, from an external electronic device or system via a communication device of the hearing device, contextual factor data indicative of one or more factors impacting a current context or wellbeing of the wearer. The method shown in FIG. 4 involves detecting 410, using a controller (or processor) of the hearing device, presence and/or absence of tinnitus of the wearer using the physiologic sensor signals, the non-physiologic sensor signals, and the contextual factor data. The method also involves detecting 412, by the controller, severity of tinnitus of the wearer using the physiologic sensor signals, the non-physiologic sensor signals, and the contextual factor data if tinnitus is present.

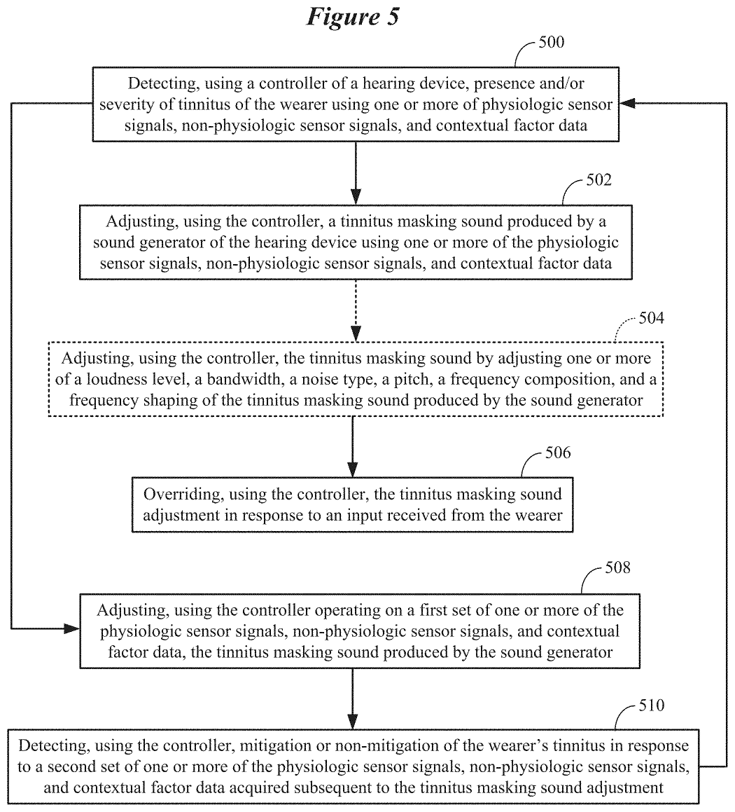

FIG. 5 illustrates a method of detecting tinnitus of a hearing device wearer and automatically adjusting a tinnitus masking sound generated by the hearing device in accordance with any of the embodiments disclosed herein. The method shown in FIG. 5 involves detecting 500, using a controller (or processor) of the hearing device, presence and/or severity of tinnitus of the wearer using one or more of physiologic sensor signals, non-physiologic sensor signals, and contextual factor data. The method also involves adjusting 502, using the controller, a tinnitus masking sound produced by a sound generator of the hearing device using one or more of the physiologic sensor signals, non-physiologic sensor signals, and contextual factor data. Adjusting the tinnitus masking sound using the controller can involve, for example, adjusting 504 one or more of a loudness level, a bandwidth, a noise level, a pitch, a frequency composition, and a frequency shaping of the tinnitus masking sound produced by the sound generator.

The method shown in FIG. 5 involves overriding 506, using the controller, the tinnitus masking sound adjustment in response to an input received from the wearer. In some cases, automatic adjustment of the tinnitus masking sound by the controller may result in a masking sound that is undesirable to the wearer (e.g., too loud, not loud enough, an unpleasant pitch). The wearer can communicate an override input to the controller, such as by way of a button push, a finger or hand gesture, or a voice command. In response to the override input, the controller can adjust the tinnitus masking sound to a previously used tinnitus masking sound or a predetermined default tinnitus masking sound, for example. In accordance with any of the embodiments disclosed herein which include a machine learning processor, adjustments to the tinnitus masking sound made by the controller can be enhanced or optimized by an algorithm implemented by the machine learning processor by analyzing tinnitus masker adjustments prior to, in response to, and subsequent to receipt of an override command initiated by the wearer and received by the machine learning processor.

The method of FIG. 5 can involve adjusting 508, using the controller operating on a first set of one or more of the physiologic sensor signals, non-physiologic sensor signals, and contextual factor data, the tinnitus masking sound produced by the sound generator. The method can also involves detecting 510, using the controller, mitigation or non-mitigation of the wearer's tinnitus in response to a second set of one or more of the physiologic sensor signals, non-physiologic sensor signals, and contextual factor data acquired subsequent to the tinnitus masking sound adjustment.

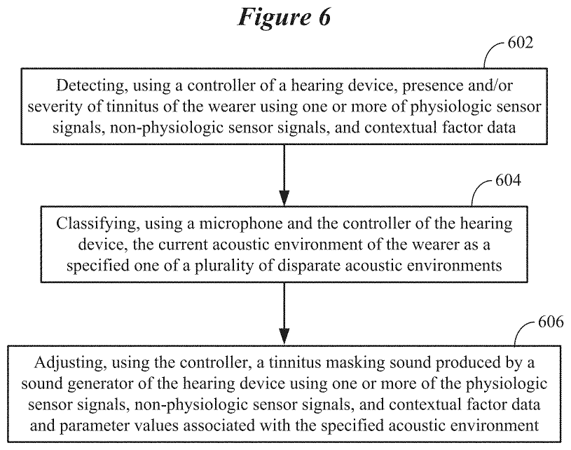

FIG. 6 illustrates a method of detecting tinnitus of a hearing device wearer and automatically adjusting a tinnitus masking sound generated by the hearing device in accordance with any of the embodiments disclosed herein. The method shown in FIG. 6 involves detecting 602, using a controller (or processor) of the hearing device, presence and/or severity of tinnitus of the wearer using one or more of physiologic sensor signals, non-physiologic sensor signals, and contextual factor data. The method also involves classifying 604, using a microphone and the controller of the hearing device, the current acoustic environment of the wearer as a specified one of a plurality of disparate acoustic environments. The method further involves adjusting 606, using the controller, a tinnitus masking sound produced by a sound generator of the hearing device using one or more of the physiologic sensor signals, non-physiologic sensor signals, and contextual factor data, and parameter values associated with the specified acoustic environment. Adjusting 606 the masking sound produced by the sound generator can comprise adjusting a frequency response of the tinnitus masker sound in frequency bands appropriate for the specified acoustic environment.

For example, the controller or other processor of the hearing device can include, or be operatively coupled to, a classification module configured to classify the current acoustic environment pf the hearing device wearer using signals received from one or more microphones of the hearing device. During operation of the hearing device within an acoustic environment, the classification module can classify the acoustic environment as at least one of a quiet environment with desired sound (e.g., a friend speaking, music playing), a loud environment with desired sound, and an environment with desired sound and wind, machine or other noise. For example, the classification module can be configured to classify sound sensed by the microphone or microphones of the hearing device as one of music, speech, and non-speech within a quiet environment or a loud environment. The non-speech sound classified by the classification module can include one or more of wind noise, machine noise, and other annoying sounds. The classification module can be implemented in accordance with the classification module embodiments disclosed in commonly owned U.S. Published Patent Application Serial No. 2011/0137656 and U.S. Application Ser. No. 62/956,824 filed on Jan. 3, 2020 under Attorney Docket No. ST0891PRV/0532.000891US60, which are incorporated herein by reference in their respective entireties.

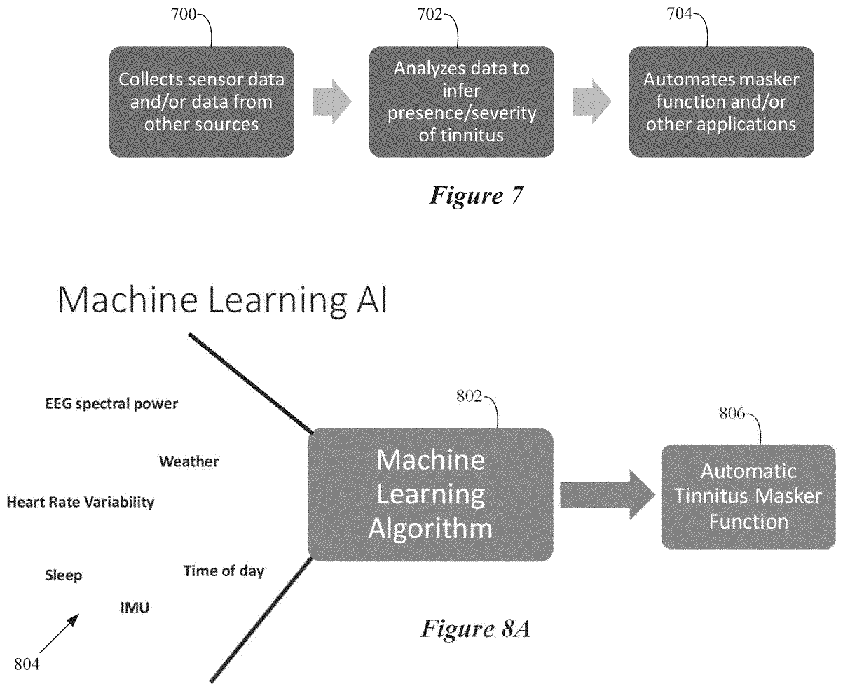

FIG. 7 illustrates a method of detecting tinnitus of a hearing device wearer and automatically adjusting a masker function and/or launching one or more applications in accordance with any of the embodiments disclosed herein. The method shown in FIG. 7 involves collecting 700 or more of physiologic sensor signals, non-physiologic sensor signals, and contextual factor data and/or data from various sources. The method also involves analyzing 702 the acquired data to infer the presence and/or severity of the wearer's tinnitus. In response to the acquired data, the controller of the hearing device automates the masker function and/or launches other applications.

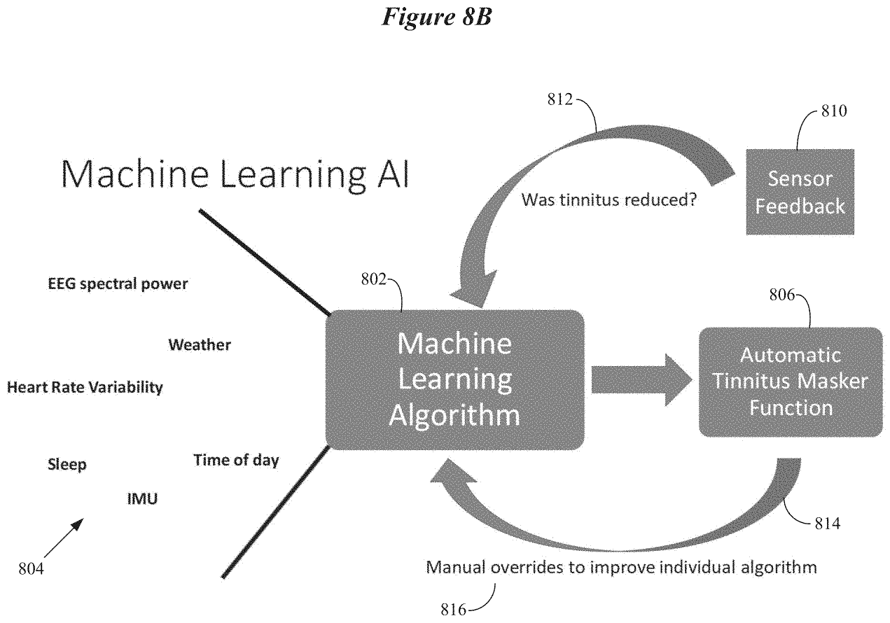

For example, FIGS. 8A and 8B illustrate aspects of machine learning by a machine learning processor (e.g., see FIG. 1A) of a hearing device configured to receive and operate on a wide variety of sensor data and contextual data in accordance with any of the embodiments disclosed herein. As was discussed previously, the machine learning processor can receive various forms of physiologic sensor data, non-physiologic sensor data, and/or contextual data. These data 804 can include one or more of EEG spectral power, heart rate variability, physiologic sleep data, motion sensor (e.g., IMU) data, time of day data, and current weather data (see, e.g., any of the data discussed above with reference to FIG. 1B). These data 804 can be input to a machine learning algorithm 802 implemented by the machine learning processor of the hearing device to inform and refine an automatic tinnitus masker function of the hearing device.

An automatic tinnitus masker function 806 can be implemented by the controller of the hearing device to treat tinnitus of the wearer when detected by the hearing device. Physiologic and non-physiologic sensor data 810 can be fed back to the machine learning processor to determine, by execution of the machine learning algorithm 802, if the wearer's tinnitus is mitigated or not mitigated (and to what extent). The machine learning algorithm 802 can be refined by the machine learning processor based on the sensor feedback 810 and in response to any manual overrides 816 initiated by the wearer via a wearer override input. The sensor feedback 810 and the manual overrides can be used to improve the wearer's individual machine learning algorithm 802.

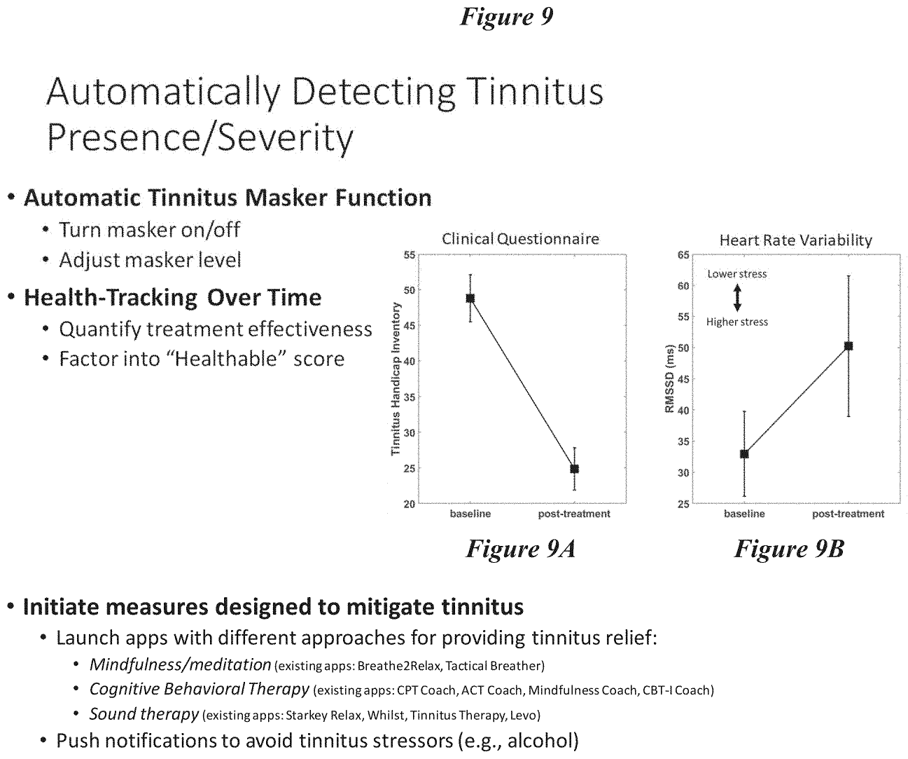

A clinical evaluation was conducted on a number of hearing aid wearers to evaluate the efficacy of a tinnitus masker function implemented by a hearing aid. The masker function of the clinical evaluation was manually activated, de-activated, and adjusted by the hearing aid user. Qualitative data (clinical questionnaire data shown in FIG. 9A) and quantitative data (HRV data shown in FIG. 9B) of the tinnitus treatment was collected. FIG. 9 provides a summary of the clinical evaluation and results. As can be seen in FIGS. 9A and 9B, the tinnitus masker function of the hearing aids provided for a significant improvement in a wearer's tinnitus when subjected to tinnitus mitigation treatment by the hearing aids (e.g., a reduced tinnitus handicapped inventory, as shown in FIG. 9A, a reduced level of stress, as shown in FIG. 9B).

FIG. 9 demonstrates that various measures can be implemented using a hearing device to detect and mitigate tinnitus of the hearing device wearer in accordance with any of the embodiments disclosed herein. FIG. 9 also shows additional capabilities of the hearing device when communicatively coupled to an external electronic device or system. For example, the hearing device 100 can be configured to communicatively couple to a smartphone or tablet which can launch one or more apps directed to different approaches to providing tinnitus relief. For example, one or more apps can be launched using the smartphone or tablet to engage the wearer in mindfulness or meditation exercises, cognitive behavioral therapy, and/or sound therapy. In response to detecting tinnitus, the hearing device 100 can transmit an alert signal to the smartphone or tablet which, in response, can notify the hearing device wearer to avoid tinnitus stressors (e.g., alcohol, caffeine, nicotine). In some implementations, the hearing device 100, alone or in cooperation with a smartphone or tablet, can be configured to provide binaural beat therapy, which has proven to be an effective treatment of tinnitus perception and tinnitus loudness. Other sound therapy can be provided by the hearing device 100 such as the sound of ocean waves, which is effective in treating stress-induced tinnitus.

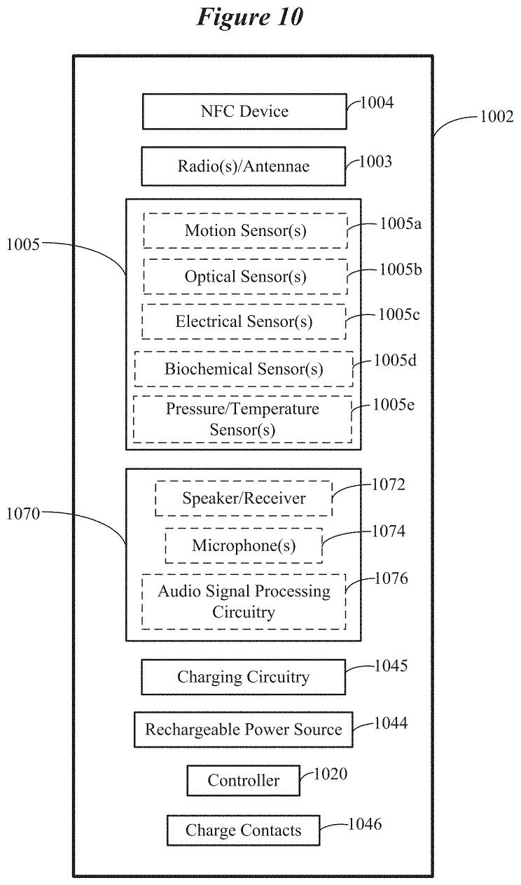

FIG. 10 is a block diagram of a representative ear-wearable electronic device 1002 which can incorporate a tinnitus detection and mitigation facility in accordance with any of the embodiments disclosed herein. The device 1002 is representative of a wide variety of electronic devices configured to be deployed in, on or about an ear of a wearer, including any of the devices discussed hereinabove. The device 1002 can include an NFC device 1004 of a type previously described, and may also include one or more RF radios/antennae 1003 (e.g., compliant with a Bluetooth® or IEEE 802.11 protocol). The RF radios/antennae 1003 can be configured to effect communications with an external electronic device, communication system, and/or the cloud. Data acquired by the ear-wearable electronic device 1002 can be communicated to a smartphone, laptop, network server, and/or the cloud (e.g., a cloud server and/or processor). The device 1002 includes a controller 1020, a rechargeable power source 1044, charging circuitry 1045, and charge contacts 1046.

The device 1002 can include one or more sensors 1005 of a type previously described in connection with the physiologic and non-physiologic sensor arrangements 150, 160. For example, the device 1002 can include one or more of a motion sensor 1005a, one or more optical physiologic and non-physiologic sensors 1005b, one or more physiologic electrode-based sensors 1005c, one or more biochemical sensors 1005d, and/or one or both of barometric and/or temperature sensors 1005e.

In accordance with any of the embodiments disclosed herein, the device 1002 can be configured as a hearing device or a hearable which includes an audio processing facility 1070. The audio processing facility 1070 includes a masking sound generator (e.g., sound generator 130) and can also include audio signal processing circuitry 1076 coupled to an acoustic transducer 1072 (e.g., sound generator 130, speaker, receiver, bone conduction device) and optionally to one or more microphones 1074 coupled to the audio signal processing circuitry 1076. In other embodiments, the device 1002 can be devoid of the one or more microphones 1074.

According to embodiments that incorporate the audio processing facility 1070, the device 1002 can be implemented as a hearing assistance device that can aid a person with impaired hearing. For example, the device 1002 can be implemented as a monaural hearing aid or a pair of devices 1002 can be implemented as a binaural hearing aid system, in which case left and right devices 1002 are deployable with corresponding left and right wearable sensor units. The monaural device 1002 or a pair of devices 1002 can be configured to effect bi-directional communication (e.g., wireless communication) of data with an external source, such as a remote server via the Internet or other communication infrastructure. The device or devices 1002 can be configured to receive streaming audio (e.g., digital audio data or files) from an electronic or digital source. Representative electronic/digital sources (e.g., accessory devices) include an assistive listening system, a streaming device (e.g., a TV streamer or audio streamer), a remote microphone, a radio, a smartphone, a laptop, a cell phone/entertainment device (CPED) or other electronic device that serves as a source of digital audio data, control and/or settings data or commands, and/or other types of data files.

The controller 1020 shown in FIG. 10 (and the controller 120 shown in FIG. 1A) can include one or more processors or other logic devices. For example, the controller 1020, 120 can be representative of any combination of one or more logic devices (e.g., multi-core processor, digital signal processor (DSP), microprocessor, programmable controller, general-purpose processor, special-purpose processor, hardware controller, software controller, a combined hardware and software device) and/or other digital logic circuitry (e.g., ASICs, FPGAs), and software/firmware configured to implement the functionality disclosed herein. The controller 1020, 120 can incorporate or be coupled to various analog components (e.g., analog front-end), ADC and DAC components, and Filters (e.g., FIR filter, Kalman filter). The controller 1020, 120 can incorporate or be coupled to memory. The memory can include one or more types of memory, including ROM, RAM, SDRAM, NVRAM, EEPROM, and FLASH, for example.

Although reference is made herein to the accompanying set of drawings that form part of this disclosure, one of at least ordinary skill in the art will appreciate that various adaptations and modifications of the embodiments described herein are within, or do not depart from, the scope of this disclosure. For example, aspects of the embodiments described herein may be combined in a variety of ways with each other. Therefore, it is to be understood that, within the scope of the appended claims, the claimed invention may be practiced other than as explicitly described herein.

All references and publications cited herein are expressly incorporated herein by reference in their entirety into this disclosure, except to the extent they may directly contradict this disclosure. Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g. 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a radio chip may be operably coupled to an antenna element to provide a radio frequency electric signal for wireless communication).

Terms related to orientation, such as “top,” “bottom,” “side,” and “end,” are used to describe relative positions of components and are not meant to limit the orientation of the embodiments contemplated. For example, an embodiment described as having a “top” and “bottom” also encompasses embodiments thereof rotated in various directions unless the content clearly dictates otherwise.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of” “consisting of,” and the like are subsumed in “comprising,” and the like. The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

What is claimed is:

1. An ear-wearable electronic device, comprising:
a housing configured to be worn in, at or about an ear of a wearer;
a sound generator disposed in the housing and configured to produce at least a tinnitus masking sound;
a physiologic sensor arrangement disposed in or supported by the housing and configured to measure a plurality of physiologic parameters or physiologic conditions of the wearer, the physiologic sensor arrangement configured to produce physiologic sensor signals in response to the physiologic sensor measurements; and
a controller operatively coupled to the sound generator and the physiologic sensor arrangement, the controller configured to detect one or more of presence, absence, and severity of tinnitus of the wearer using the physiologic sensor signals.

2. The device of claim 1, wherein the device is devoid of a microphone.

3. The device of claim 1, wherein:
the physiologic sensor arrangement comprises a sensor configured to produce an electroencephalography (EEG) signal; and
the controller is configured to:
produce EEG spectral power data using the EEG signal; and
detect one or more of absence, presence, and severity of tinnitus of the wearer using the EEG spectral power data.

4. The device of claim 1, wherein:
the physiologic sensor arrangement comprises a sensor configured to produce an electrocardiogram (ECG) signal; and
the controller is configured to:
produce heart rate variability data using the ECG signal; and
detect one or more of absence, presence, and severity of tinnitus of the wearer using the heart rate variability data.

5. The device of claim 1, wherein:
the physiologic sensor arrangement comprises a sensor configured to produce a photoplethysmography (PPG) signal; and
the controller is configured to:
produce heart rate variability data using the PPG signal; and
detect one or more of absence, presence, and severity of tinnitus of the wearer using the heart rate variability data.

6. The device of claim 1, wherein the physiologic sensor arrangement comprises a sensor configured to produce one or both of an electromyography (EMG) signal and an electrooculography (EOG) signal.

7. The device of claim 1, wherein the physiologic sensor arrangement comprises a sensor configured to produce one or both of an electrodermal activity signal and a galvanic skin response signal.

8. The device of claim 1, wherein the physiologic sensor arrangement comprises a biochemical sensor configured to one or both of sense changes in blood serotonin levels of the wearer and sense changes in blood glucose levels of the wearer.

9. The device of claim 1, comprising:
a motion sensor disposed in or supported by the housing and coupled to the controller, the motion sensor configured to generate a motion sensor signal indicative of wearer motion;
wherein the controller is configured to detect one or more of absence, presence, and severity of tinnitus of the wearer using the motion sensor signal.

10. The device of claim 1, comprising:
an optical sensor supported by the housing and coupled to the controller, the optical sensor configured to generate an optical sensor signal indicative of ambient light intensity;
wherein the controller is configured to detect one or more of absence, presence, and severity of tinnitus of the wearer using the optical sensor signal.

11. The device of claim 1, comprising:
a microphone arrangement supported by the housing and coupled to the controller, the microphone arrangement comprising one or more microphones configured to generate a microphone signal indicative of sound within the wearer's current acoustic environment;
wherein the controller is configured to detect one or more of absence, presence, and severity of tinnitus of the wearer using the microphone signal.

12. The device of claim 1, comprising:
a barometric pressure sensor supported by the housing and coupled to the controller, the barometric pressure sensor configured to generate a pressure sensor signal indicative of ambient barometric pressure;
wherein the controller is configured to detect one or more of absence, presence, and severity of tinnitus of the wearer using the pressure sensor signal.

13. The device of claim 1, wherein the controller is configured to adjust the tinnitus masking sound produced by the sound generator by producing a tinnitus masking sound that substantially matches the wearer's tinnitus.

14. The device of claim 1, wherein:
the controller is configured to adjust the tinnitus masking sound produced by the sound generator using the physiologic sensor signals; and
the physiologic sensor signals are produced by two or more of an EEG sensor, an ECG sensor, an EMG sensor, an EOG sensor, a PPG sensor, an electrodermal activity sensor, a GSR sensor, and a biochemical sensor.

15. The device of claim 1, wherein the controller is configured to adjust one or more of a loudness level, a bandwidth, a noise type, a pitch, a frequency composition, and a frequency shaping of the tinnitus masking sound produced by the sound generator.

16. The device of claim 1, comprising:
a microphone arrangement supported by the housing and coupled to the controller, the microphone arrangement comprising one or more microphones configured to generate a microphone signal indicative of sound within the wearer's current acoustic environment;
wherein the controller is configured to:
classify the current acoustic environment of the wearer as a specified one of a plurality of disparate acoustic environments; and
adjust the tinnitus masking sound produced by the sound generator using the physiologic sensor signals and parameter values associated with the specified acoustic environment.

17. The device of claim 1, comprising:
a non-physiologic sensor arrangement comprising one or more non-physiologic sensors configured to sense a least one non-physiologic parameter or condition impacting a current context or wellbeing of the wearer, the non-physiologic sensor arrangement configured to produce non-physiologic sensor signals in response to the non-physiologic sensor measurement; and
wherein the controller is configured to detect one or more of presence, absence, and severity of tinnitus of the wearer using the physiologic sensor signals and the non-physiologic sensor signals.

18. The device of claim 17, comprising:
a communication device disposed in or supported by the housing and configured to wirelessly communicate with an external electronic device or system and to receive, from the external electronic device or system, contextual factor data indicative of one or more factors impacting a current context or wellbeing of the wearer;
wherein the controller is configured to detect one or more of presence, absence, and severity of tinnitus of the wearer using the physiologic sensor signals, the non-physiologic sensor signals, and the contextual factor data.

19. The device of claim 18, wherein the contextual factor data comprises one or more of time of day data, local weather data, wearer sleep data, data indicative of the wearer's nutrition, wearer stress data, wearer medication data.

20. The device of claim 18, wherein the controller comprises, or is operatively coupled to, a processor configured with instructions to process one or more of the physiologic sensor signals, the non-physiologic sensor signals, and the contextual factor data via a machine learning algorithm to adjust the tinnitus masking sound produced by the sound generator.

21. The device of claim 18, comprising:
a microphone arrangement supported by the housing and coupled to the controller, the microphone arrangement comprising one or more microphones configured to generate a microphone signal indicative of sound within the wearer's current acoustic environment;
wherein the controller comprises, or is operatively coupled to, a processor configured with instructions to:
classify, via a first neural network, the acoustic environment of the wearer as a specified one of a plurality of disparate acoustic environments; and
process one or more of the physiologic sensor signals, the non-physiologic sensor signals, and the contextual factor data, via a second neural network, to adjust the tinnitus masking sound produced by the sound generator using the one or more of the physiologic sensor signals, the non-physiologic sensor signals, the contextual factor data, and parameter values associated with the specified acoustic environment.

22. The device according to claim 18, wherein the controller comprises, or is operatively coupled to, a processor configured with instructions to:
process first data comprising one or more of the physiologic sensor signals, the non-physiologic sensor signals, and the contextual factor data via a machine learning algorithm to adjust the tinnitus masking sound produced by the sound generator; and
detect mitigation or non-mitigation of the wearer's tinnitus, via the neural network, in response to second data comprising one or more of the physiologic sensor signals, the non-physiologic sensor signals, and the contextual factor data acquired subsequent to the tinnitus masking sound adjustment.

23. A method implemented by an ear-wearable electronic device worn by a wearer, comprising:
measuring, using a physiologic sensor arrangement of the device, a plurality of one or both of physiologic parameters and physiologic conditions of the wearer;
producing, by the physiologic sensor arrangement, physiologic sensor signals in response to the physiologic sensor measurements; and
detecting, using a controller of the device, one or more of presence, absence and severity of tinnitus of the wearer using the physiologic sensor signals.

24. The method of claim 23, comprising:
measuring, using a non-physiologic sensor arrangement of the device, at least one non-physiologic parameter or at least one condition impacting a current context or wellbeing of the wearer;
producing, by the non-physiologic sensor arrangement, non-physiologic sensor signals in response to the non-physiologic sensor measurement; and
detecting, using the controller, one or more of presence, absence, and severity of tinnitus of the wearer using the physiologic sensor signals and the non-physiologic sensor signals.

25. The method of claim 24, comprising:
receiving, from an external electronic device or system, contextual factor data indicative of one or more factors impacting a current context or wellbeing of the wearer; and
detecting, using the controller, one or more of presence, absence, and severity of tinnitus of the wearer using the physiologic sensor signals, the non-physiologic sensor signals, and the contextual factor data.
